(19) United States
(12) Plant Patent Application Publication
(10) Pub. No.: US 2003/0019001 P1
(43) Pub. Date: Jan. 23, 2003

(54) Nandina domestica 'Jaytee'

(76) Inventor: Ralph C. Rushing, Semmes, AL (US)

Correspondence Address: Ralph C. Rushing, 3220 Redstone Drive West, Semmes, AL, 36575, US

(21) Appl. No.: 09/893,950

(22) Filed: Jun. 29, 2001

(51) Int. Cl. A01H 5/00

(52) U.S. Cl. PLT/235

(57) ABSTRACT

‘Jaytee’, a new and distinct variety of Nandina domestica, which exhibits new, improved, and superior characteristics not known to exist in other varieties of Nandina domestica. These characteristics include rapid, aggressive growth, abundant fruit and flowers, and unique leaf coloration. This plant has a rhizomatous growth habit, that results in a compact, dense plant with no tendency to legginess.

### CROSS REFERENCE TO RELATED APPLICATION

none

### STATEMENT AS TO RIGHTS TO INVENTION MADE UNDER FEDERALLY-SPONSORED RESEARCH AND DEVELOPMENT

none

### BACKGROUND OF THE INVENTION

(1) Field of Invention

The present invention relates to the discovery and asexual reproduction of a new and distinct cultivar of Nandina domestica, and hereinafter referred to by the cultivar name ‘Jaytee.’

(2) Description of relevant prior art including information disclosed under 37 CFR 1.971.99.

This new variety was discovered in a cultivated bed of Nandina domestica seedling plants that were growing at Rushing Nursery, Inc. in Semmes, Alabama. The new variety of Nandina domestica has been named ‘Jaytee.’ ‘Jaytee’ berries more abundantly and has a denser habit and is more rhizomatous than ‘Harbour Dwarf.’ ‘Jaytee’ also exhibits leaf colors that have a broader color spectrum than ‘Harbour Dwarf.’

Both tissue culture and stem propagation have demonstrated that this new Nandina is stable in maintaining its unique characteristics.

### SUMMARY OF THE INVENTION

The ‘Jaytee’ variety has been observed and extensively tested in different growing situations for five years. Its recognizable characteristics, which distinguish it from other known Nandina cultivars are:

Compact, dense form, spreading by rhizomes, with a mature height of 18-22″

Aggressive, vigorous growth

Has proven adaptable to harsh environmental conditions

Abundant flowers and fruit at a relative early age of 1½ years

Exhibits unique leaf coloration.

New growth is dark, reddish brown

Intermediate growth is light green with reddish hues

Mature winter foliage is purple with pinkish hues and dark green with yellow tint

Leaves are large and appear thick and heavily textured

Is sun and shade tolerant maintaining its proportions equally well in both conditions

Versatile uses are: groundcover, mass plantings, and individual specimens

Has shown no susceptibility to mosaic virus

Has survived −6 degrees in Zone 6. Winter hardiness testing is being continued.

Has proved to be adaptable to extreme landscape conditions, where ‘Harbour Dwarf’ would have difficulty in becoming established.

Comparison—‘Jaytee’ and ‘Harbour Dwarf’

| | Applicant (‘JayTee’) | Harbour Dwarf |
|---|---|---|
| Height | 18-22″ | 27-36″ |
| | | (taller in heavy shade) |
| Width | 30-36″ | 24″ |
| Leaf Length | 30-34 mm | 28-30 mm |
| Leaf Width | 18-20 mm | 16-18 mm |
| Leaflet Length | 5.5-7 mm | 5-6 mm |
| Leaflet Width | 2.5-3 mm | 1.5-2 mm |
| Foliage | Opposite, bipinnate, | Opposite, bi-tripinnate, |
| | compound evergreen | compound evergreen |
| New Foliage | Dark, reddish brown | Light green with pinkish |
| Color | (RHS 174A) | hues (RHS 143A) |
| Intermediate | Light green with reddish | Light yellowish-green |
| Foliage Color | hues (RHS 177A) | (RHS 144A) |
| Mature Foliage | Dark green (RHS 136A) | Dark green (RHS 139A) |
| Color | | |
| Winter Foliage | Purple with pinkish hues | Orange to bronzy red to |
| Color | (RHS N187A) and Dark | reddish purple (RHS 173) |
| | greenish with yellow tint | |
| | (RHS 147A) | |
| Foliage Texture | leathery in appearance | somewhat leathery in |
| | | appearance |
| Rachis Color | Reddish-purple (RHS 71A) | Brownish-orange |
| | | (RHS 176B) |
| Flowers | Numerous perfect flowers | Occasionally flowers with |
| | that are slightly pink in | perfect flowers that are |
| | bud, turning white with | slightly pink in bud, turning |
| | yellow antlers. Flowers | white with yellow antlers. |
| | develop in May-June. | |
| Fruit | Abundant clusters of red | Few small red berries, |
| | berries (1/4″ in diameter). | sparingly produced. |
| | Typically larger than those | |
| | of ‘Harbour Dwarf.’ | |
| Growth Habit | Dense, dwarf, compact | Dwarf form, stiff wide- |
| | mounding form. | spreading crown, becoming |
| | Aggressive, vigorous, | more graceful in |
| | spreading by rhizomes. | appearance with age. |
| | Maintains fullness with no | Tendency towards legginess |
| | tendancy toward legginess. | in shade. |
| Disease | no mosaic virus | Susceptible to mosaic |
| | susceptibility to date | virus |

Comparison—‘Jaytee’ and other Nandina cultivars

| | Mature Size | Other Characteristics |
|---|---|---|
| Jaytee | 18-22″ × 30-36″ | as previously described |
| Heavenly | 6-8′ H | perennating by rhizomes, prone to |
| Bamboo | | legginess, generally fruits abundantly |
| Compacta | 4-6′ H | perennating by rhizomes, fruiting |
| | | capability dependent on selection |
| Moonbay | 3-3½′ H | not rhizomatous, flowering sparse to nil |
| Gulf Stream | 3-3½′ H | not rhizomatous, flowering sparse to nil |
| Lowboy | 5′ H × 3.5′ W | typical species leaf coloration, |
| | | moderately rhizomatous, good fruit set |

### BRIEF DESCRIPTION OF THE DRAWINGS

FIG. 1-A is a color photograph of the original plant ‘Jaytee’ growing in a landscape setting. The original plant has never been pruned and is 18″ in height at 6 years of age.

FIG. 1-B is a photograph of the new variety growing in a 20 gallon container. This photo shows how rhizomatous this plant is.

FIG. 2-A is a side by side comparison of the new variety, shown on the right, with a ‘Harbour Dwarf’ Nandina, shown on the left. Both plants are in a 3 gallon container at approximately 16 months of age.

FIG. 2-B represents the same plants as FIG. 2-A, but as a top view. ‘Jaytee’ on the right and ‘Harbour Dwarf’ on the left.

FIG. 3-A is a side by side comparison of the new variety's leaf color progression. From left to right are new growth, intermediate growth (2), and mature growth.

FIG. 3-B is a photograph of the new variety in a landscape setting. This photo shows the plants unique leaf color in the summer.

FIG. 4-A is a photograph of the new variety's fruit.

FIG. 4-B is a photograph of the new variety's leathery leaf texture. It also shows the reddish-brown new growth.

FIG. 5-A is a photograph showing the new variety's flower buds.

FIG. 5-B is a photograph of the new variety's mature winter color.

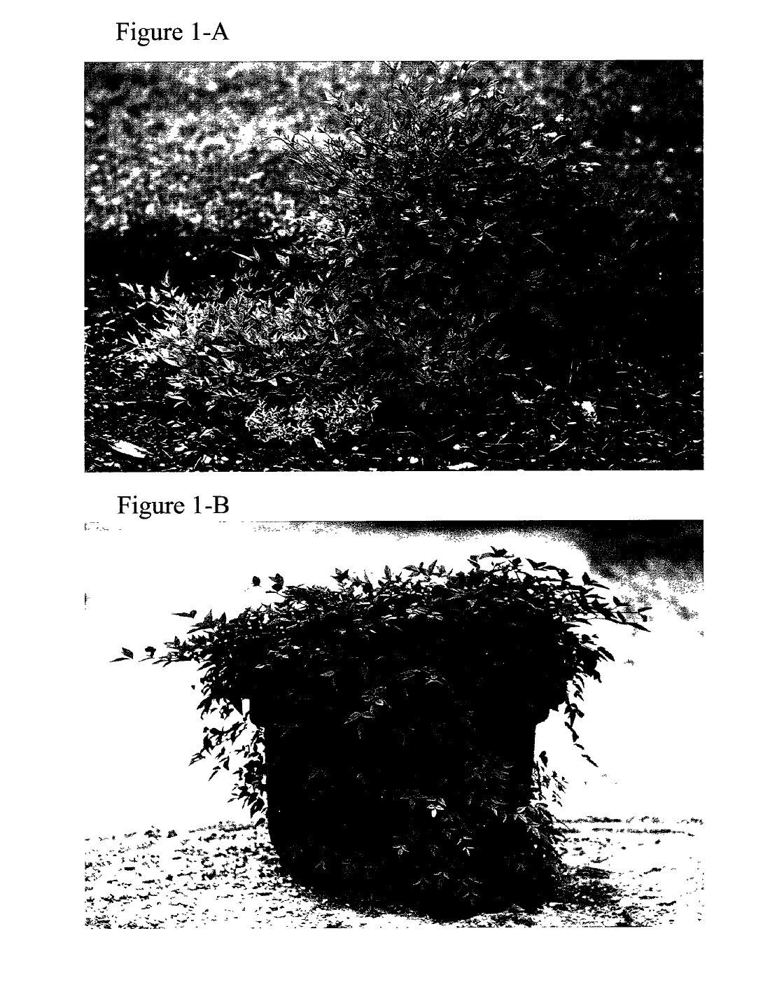

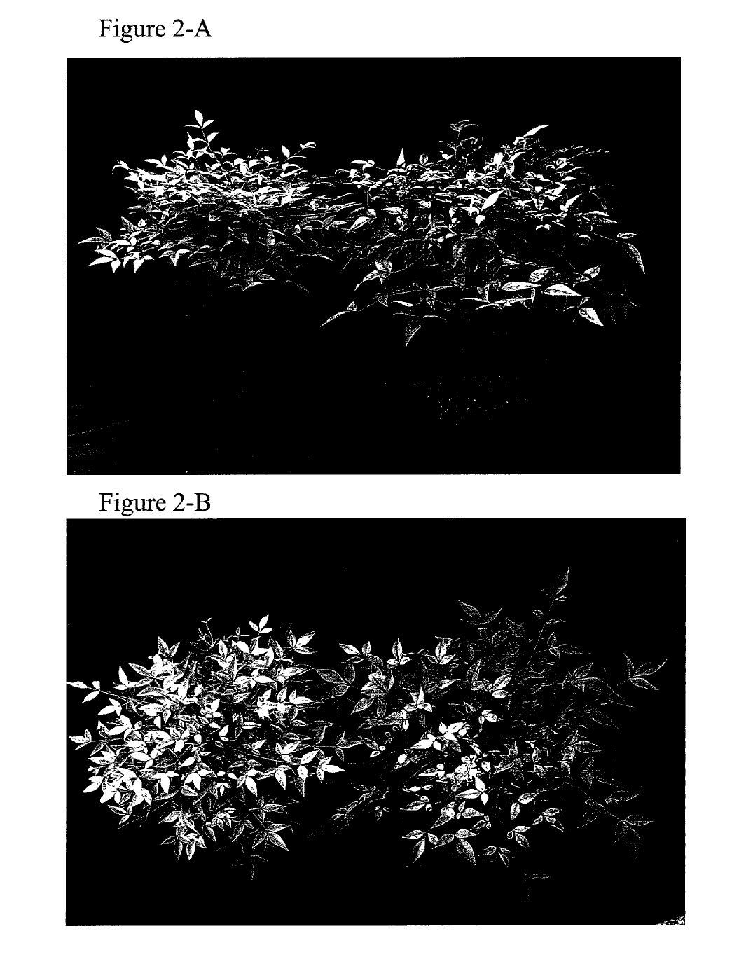

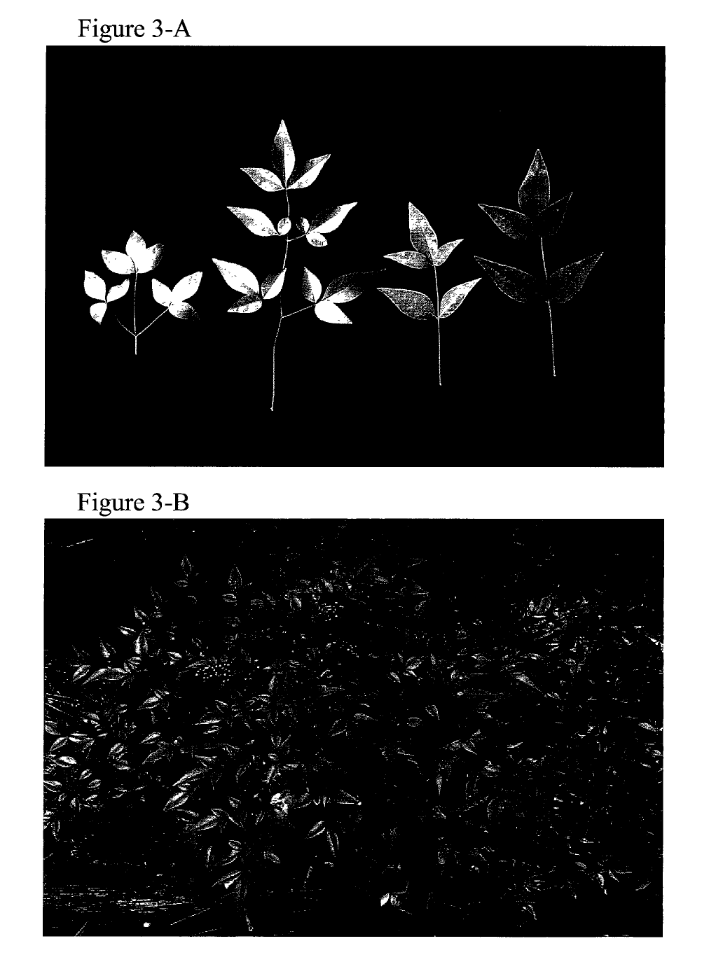

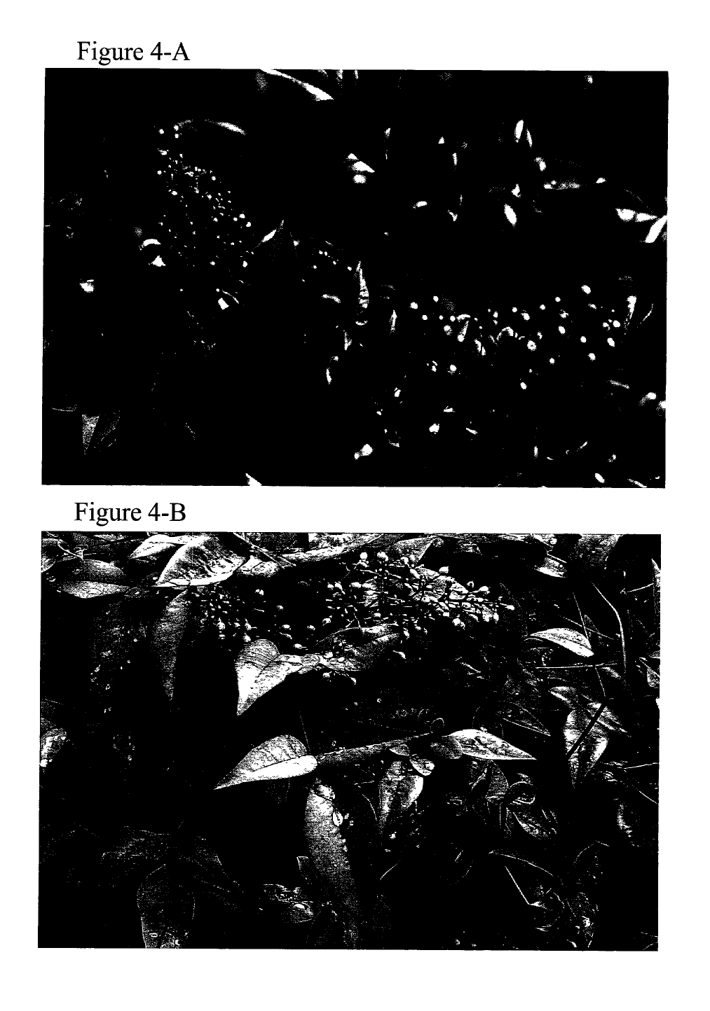

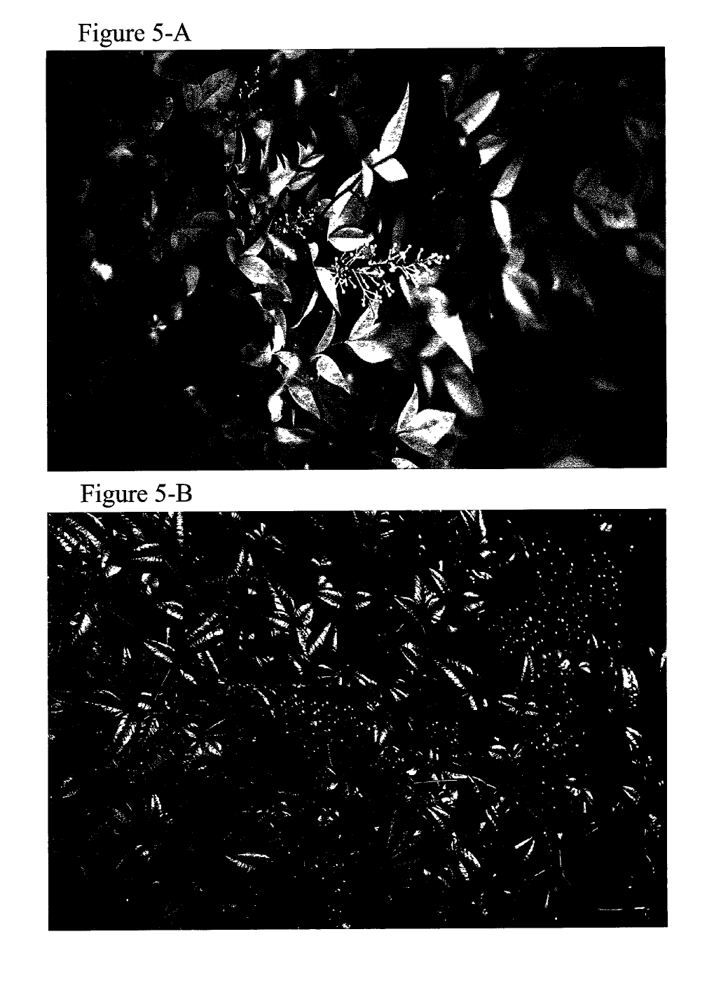

### BOTANICAL DESCRIPTION OF THE PLANT

Name: Nandina domestica ‘Jaytee.’

Parentage: A chance seedling in a population of Nandina domestica

Size: Ultimate mature size at 24-36 months is 18-22″ height × 30-36″ width.

Growth Habit: Dwarf, dense, compact mounding form. Aggressive and vigorous in growth. Rhizomatous spreading habit.

Foliage:

Arrangement.—Opposite, bipinnately compound, evergreen

Leaflet base.—Obtuse

Leaflet tips.—Acuminte

Leaflet shape.—Ovate-Lanceolate

Leaflet margin.—Entire

Leaflet length.—5.5-7 mm

Leaflet width.—2.5-3 mm

Leaf length.—30-34 mm

Leaf width.—18-20 mm

Stem form.—Upright

Stem texture.—Smooth, longitudinally ridged

Leaflet texture.—Leathery appearance

Leaflet color.—Young leaflets are closest to RHS 1 74A-dark brownish red. Intermediate leaflets are closest to RHS 177A-very light green with reddish hues. Medium leaflets are closest to RHS 139A-medium green. Mature leaflets are closest to RHS 136A-dark green. Winter color is RHS-N187A-purple with pinkish hues and RHS 147A-dark greenish with yellow tint.

Flowers: Numerous perfect flowers that are slightly pink in bud, turning white with yellow antlers. Panicle formation of flowers. Panicles are 12-18 mm long. Flowers appear at 1½ years of age. Creamy White-RHS 155A.

Natural Flowering Season: May-June

Fruit: Abundant clusters of red berries, ca. 1/4″ diameter, two-seeded

Climatic Range: This plant has survived winter temperatures as low as −6 degrees F. in Shrewsbury, Pa. (Zone 6).

Disease Resistance: Has shown no susceptibility to mosaic virus

1. The present plant is readily distinguishable from other Nandina cultivars as the Nandina described herein does not have the same characteristics as other Nandina in coloring, growth habit, or fruit bearing.
